(19) United States
(12) Patent Application Publication
(10) Pub. No.: US 2012/0179232 A1
(43) Pub. Date: Jul. 12, 2012

(54) HEATING DEVICE

(75) Inventor: Atsushi Matsuo, Ibaraki-shi (JP)

(73) Assignee: KOBAYASHI PHARMACEUTICAL CO., LTD., Osaka-shi, Osaka (JP)

(21) Appl. No.: 13/386,129

(22) PCT Filed: Dec. 4, 2009
(86) PCT No.: PCT/JP2009/070433

§ 371 Date: Jan. 20, 2012

(30) Foreign Application Priority Data
Sep. 29, 2009 (JP) 2009-224311

(51) Int. Cl. A61F 7/08

(52) U.S. Cl. 607/109; 607/114

(57) ABSTRACT

An object of the invention is to provide a heating device that can warm the body to promote blood circulation, while restricting the movement of an area of the body to which the heating device is applied.

A heating device attached to a body, comprising:
a heat generating portion having an exothermic composition enclosed in one or more sections, the exothermic composition generating heat by contact with air; and
at least one band portion formed of an extensible material and attached to the heat-generating portion;
the exothermic composition accommodated in each section having a weight per unit area of 0.11 to 0.94 g/cm²; and
each section having an area of 15 to 150 cm².

### TECHNICAL FIELD

The present invention relates to a heating device attached to the body.

### BACKGROUND ART

Methods for treating joint pain or the like by bringing a heating device into contact with an affected area to provide a heating effect have been known. Also known are methods for treating joint pain by immobilizing a joint with a plaster cast or the like to restrict forcible movement of the joint.

One example of a known method for treating joint pain by a heating effect is a method wherein a supporter having a chemical warmer accommodated in bags attached to the supporter is applied to an affected area, such as the knee joint (Patent Literature 1). Another suggested example is a thermotherapy device for use on a joint that can maintain a temperature suitable for thermotherapy for many hours, is held in place even when subjected to the movement of the joint, and that can hold the heat-generating potion in an appropriate area (Patent Literature 2). These therapeutic devices can be expected to promote blood circulation by warming an affected area, such as a joint. However, these devices cannot be expected to enhance the therapeutic effect by restricting the movement of a joint.

Additionally, chemical warmers that are directly contacted with the skin are known. By reducing the air permeability, these chemical warmers can be used as chemical warmers for direct application to the skin over an area wide enough for general use. These chemical warmers are typically attached to the body with an adhesive material (for example, Patent Literature 3). In this case, however, the use of an adhesive material to adhere a chemical warmer to the skin impairs the flexibility of the chemical warmer, such that the chemical warmer cannot fit along a joint. This chemical warmer cannot also be expected to restrict the movement of the joint.

Further, a chemical warmer that is directly contacted with the skin excessively elevates the sensible temperature if the area of the heat-generating portion in contact with the skin is too large. Thus, the area of each of the sections that accommodate an exothermic composition tends to be small (for example, Patent Literature 4). Because the area of each of the sections that accommodate the exothermic composition is small, these chemical warmers cannot be expected to enhance the therapeutic effect by restricting the movement of a joint.

### CITATION LIST

### Patent Literature

[PTL 1] Japanese Unexamined Utility Model Publication No. 1989-62820

[PTL 2] Japanese Unexamined Patent Publication No. 2007-14792

[PTL 3] Japanese Unexamined Patent Publication No. 2005-27916

[PTL 4] Japanese Unexamined Patent Publication No. 2002-514103

### SUMMARY OF INVENTION

### Technical Problem

A principal object of the invention is to provide a heating device that can warm the body to promote blood circulation, while restricting the movement of an area of the body to which the heating device is applied.

### Solution to Problem

The inventors conducted extensive research in order to promote the prevention, amelioration, and the like of joint pain by using a heating device that can promote blood circulation. Consequently, the inventors found that, by setting the weight per unit area of an exothermic composition in one or more sections that accommodate the exothermic composition within a specific range, and by setting the area of each section that accommodates the exothermic composition within a specific range, it is possible to increase the flexibility of the heating device during initial use, allow the heat-generating portion to fit along the body, and effectively immobilize the body during later use by the hardening of the heat-generating portion, thereby enhancing the prevention, amelioration, and the like of joint pain and the like. The invention was accomplished as a result of further research based on these findings. The invention relates to a heating device, a treatment method, and use of the heating device as defined in the following Items 1 to 9.

Item 1. A heating device attached to a body, comprising:

an exothermic composition that generates heat by contact with air;

a heat-generating portion that encloses the exothermic composition in one or more sections; and

at least one band portion formed of an extensible material and attached to the heat-generating portion;

the exothermic composition accommodated in each section having a weight per unit area of 0.11 to 0.94 g/cm²; and

each section having an area of 15 to 150 cm².

Item 2. The heating device according to Item 1, wherein the number of the one or more sections of the heat-generating portion is one, or two to four per area to which the heating device is applied, and the two to four sections are disposed adjacent to one another.

Item 3. The heating device according to Item 1 or 2, wherein the exothermic composition accommodated in each section has a weight per unit area of 0.15 to 0.71 g/cm², and each section has an area of 24 to 117 cm².

Item 4. The heating device according to any one of Items 1 to 3, wherein the band portion has extensibility such that a force required to elongate the band portion 150% is 115 N or less.

Item 5. The heating device according to any one of Items 1 to 4, which is applied to any of a wrist joint, ankle joint, knee joint, neck, elbow, finger joint, or waist.

Item 6. The heating device according to any one of Items 1 to 5, which is used for the treatment of joint pain or lumbago.

Item 7. The heating device according to any one of Items 1 to 6, which is used for the treatment of joint pain or lumbago by covering a wrist joint, ankle joint, knee joint, neck, elbow, finger joint, or waist.

Item 8. A method for treating joint pain or lumbago comprising covering a wrist joint, ankle joint, knee joint, neck, elbow, finger joint, or waist with the heating device of any one of Items 1 to 7, and immobilizing the joint or waist with the heat-generating portion that has hardened by oxidation of the exothermic composition.

Item 9. Use of the heating device of any one of Items 1 to 7, for manufacturing a device for treating joint pain or lumbago.

The heating device of the invention is a heating device attached to the body. The heating device includes an exothermic composition that generates heat by contact with air; a heat-generating portion that encloses the exothermic composition in one or more sections; and at least one band portion formed of an extensible material and attached to the heat-generating portion. The exothermic composition accommodated in each section has a weight per unit area of 0.11 to 0.94 g/cm², and each section has an area of 15 to 150 cm².

A specific structure of the heating device of the invention is described below.

1. Heat-Generating Portion

The heat-generating portion encloses an exothermic composition that generates heat by contact with air in one or more sections. Specifically, in the heat-generating portion, the exothermic composition is sandwiched between a first sheet (a layer that is contacted with the body) and a second sheet (a layer that is not contacted with the body) described below. The exothermic composition is thus enclosed (sealed) in the one or more sections.

The heat-generating portion may have any shape that can be advantageously brought into close contact with the body, but preferably has a rectangular shape in which the sections can be arranged in the longitudinal direction.

The size of the heat-generating portion may be suitably determined according to the size of the area to which the heating device is applied. For example, when the heat-generating portion is rectangular for use on the wrist or ankle, it is preferable that the long side be about 5 to 32 cm, and the short side be about 3 to 20 cm.

When the heat-generating portion is rectangular for use on the knee, it is preferable that the long side be about 10 to 50 cm, and the short side be about 5 to 20 cm.

When the heat-generating portion is rectangular for use on the waist, it is preferable that the long side be about 10 to 50 cm, and the short side be about 5 to 20 cm.

When the heat-generating portion is rectangular for use on the neck, it is preferable that the long side be about 10 to 30 cm, and the short side be about 3 to 10 cm.

When the heat-generating portion is rectangular for use on the elbow, it is preferable that the long side be about 10 to 30 cm, and the short side be about 5 to 10 cm.

When the heat-generating portion is rectangular for use on a finger, it is preferable that the long side be about 5 to 15 cm, and the short side be about 3 to 10 cm.

1-1. Sections

In the heat-generating portion, the number of sections in which the exothermic composition is enclosed depends on the area to which the heating device is applied. Typically, the number of sections of the heat-generating portion is preferably one, or two to four per area to which the heating device is applied, and the two to four sections are disposed adjacent to one another. For example, when the heating device is applied to the wrist or ankle, the number of sections is preferably one or two, and particularly preferably two. When the heating device is applied to the knee, the number of sections is preferably one or two, and particularly preferably two. When the heating device is applied to the waist, the number of sections is preferably two or four, and particularly preferably four. When the heating device is applied to the neck, the number of sections is preferably one or two, and particularly preferably two. When the heating device is applied to the elbow, the number of sections is preferably one or two, and particularly preferably two. When the heating device is applied to a finger, the number of sections is preferably two or four, and particularly preferably four.

Reducing the number of sections in which the exothermic composition is enclosed can further enhance the effect of restricting the movement of the area to which the heating device is applied, which is attained by adjusting the area of each section and the weight per unit area of the exothermic composition, as described below. Therefore, the prevention of pain and recovery from the pain can be promoted.

Moreover, because the exothermic composition is enclosed (sealed) in each section of the heat-generating portion in the heating device of the invention, unlike in heating devices wherein chemical warmers are accommodated in bags attached to supporters, the heat-generating portion does not move in bags in which it is accommodated, as demonstrated in, for example, Test Example 6 below. This enables the heat-generating portion to reliably fit along the area to which the heating device is applied, even during later use.

Furthermore, the size of the heating device of the invention can be adjusted using the band portion. Thus, the heating device can be readily wound around the area to which it is applied, according to the size of the user's joint or waist, as compared to heating devices having a fixed size such as supporters having a heat-generating portion.

The shape of each section is not limited, and may be suitably selected according to the shape of the area to which the heating device is applied. Each section may, for example, be rectangular, circular, or oval.

The size of each section may be adjusted within the above-defined range of the area of each section. For example, when the heat-generating portion has a rectangular shape in which rectangular sections are arranged in the longitudinal direction, the length of each section (along the short side of the heat-generating portion) is preferably about 3 to 12 cm, and more preferably about 5 to 10 cm; and the width of each section (along the long side of the heat-generating portion) is preferably about 5 to 20 cm, and more preferably about 5 to 15 cm.

The distance between adjacent sections is not limited, and may be suitably determined according to the size and the like of the heat-generating portion. Preferably, the distance between adjacent sections is about 0.5 to 20 cm, and more preferably about 0.5 to 10 cm. The distance between adjacent sections herein represents the shortest distance between a pair of the closest adjacent sections.

As stated above, in the heating device of the invention, the weight per unit area of the exothermic composition accommodated in each section is 0.11 to 0.94 g/cm²; and the area of each section is 15 to 150 cm². In order to improve the feeling of use during initial use, and prevent the area to which the heating device is applied from forcible movement during later use, it is more preferable that the weight per unit area of the exothermic composition accommodated in each section be 0.15 to 0.71 g/cm²; and that the area of each section be 24 to 117 cm². It is even more preferable that the weight per unit area of the exothermic composition accommodated in each section be 0.20 to 0.60 g/cm², and the area of each section be 32 to 80 cm².

As referred to herein, the term “the weight per unit area of the exothermic composition” accommodated in each section herein means the value obtained by dividing the weight (g) of the exothermic composition enclosed in each section by the area (cm²) of the face of each section provided in the heat-generating portion that is brought into contact with the body.

As referred to herein, the term “initial use” means the period from the time when the use of the heating device is begun by contacting the exothermic composition with air, to the time when the exothermic composition has hardened to some degree. The time from the start of heat generation of the exothermic composition to the completion of the heat generation (i.e., the heating time) varies depending on the shape of the heating device and the weight per unit area of the exothermic composition. The term “initial use” refers to, for example, a period within about the initial one-third to the initial half of the period from the time when the use of the heating device is begun by contacting the exothermic composition with air, to the time when the exothermic composition has completed the generation of heat. For example, in the case of a heating device with a heating time of 12 hours, the initial use corresponds to a period of about 0 to 4 hours to about 0 to 6 hours.

Moreover, the term “later use” means the period from the time when the exothermic composition has hardened to some degree to the time when the exothermic reaction has substantially completed, such that the heating effect can no longer be obtained. The heating time varies depending on the shape of the heating device and the weight per unit area of the exothermic composition. The term “later use” refers to, for example, a period after about the last one-third to last half of the period from the time when the use of the heating device is begun by contacting the exothermic composition with air, to the time when the exothermic composition has completed the generation of heat. For example, in the case of a heating device with a heating time of 12 hours, the later use corresponds to a period of about 8 to 12 hours to about 6 to 12 hours.

The thickness of each section in which the exothermic composition of the invention is enclosed is typically about 3 to 15 mm, preferably about 3 to 10 mm, and more preferably about 3 to 7 mm, as measured with the exothermic composition being uniformly enclosed in the section.

1-2. First Sheet (Layer Contacted with the Body)

The first sheet is not limited, and may be any film or sheet that is generally used as a packaging material of chemical warmers. A single or laminated film or sheet is used alone or in combination with a woven or nonwoven fabric. The first sheet may be either elastic or inelastic, and may be either breathable or non-breathable.

The term “inelastic” as referred to herein does not necessarily mean that the material is completely non-extensible by application of any force, but that the material may have elasticity lower than that of the extensible band portion. The term “breathable” means that the material allows air to pass through. The material may also have moisture permeability.

Typically, a thermoplastic synthetic resin or the like is used as the resin that forms the film or sheet of the first sheet. Specific examples of preferable thermoplastic synthetic resins include polyethylenes, polypropylenes, polyesters, polyamides, polyvinyl alcohols, polyvinyl chloride, polyvinylidene chloride, polyurethanes, polystyrenes, ethylene-vinyl acetate copolymer, polycarbonates, and rubber hydrochloride. These resins are used alone or in combination. Polyethylenes are particularly preferable as the resin that forms the film.

When a laminated film or sheet is used as the first sheet, it is typically produced by lamination or other methods. Lamination may be performed according to any known method. For example, lamination may be performed by a method using thermal bonding, or an adhesive such as a hot melt adhesive or an acrylic or urethane adhesive. The surfaces of the laminated film or sheet may be entirely bonded, or partially bonded to maintain flexibility.

Examples of nonwoven fabrics that may be laminated to the above-mentioned film or sheet include nonwoven fabrics containing synthetic fibers such as nylon, vinylon, polyesters, polyethylene terephthalate, rayon, acetate, acrylics, polyethylenes, polypropylenes, and polyvinyl chloride; and nonwoven fabrics containing natural fibers such as cotton, hemp, and silk. When a nonwoven fabric is laminated to the film or sheet, polyethylene terephthalate is particularly preferable as the nonwoven fabric. The nonwoven fabric may have a weight of about 20 to 100 g/m².

1-3. Second Sheet (Layer Not Contacted with the Body)

The second sheet may be a film or sheet that is breathable on all of the surfaces thereof. Typically, a single or laminated porous film or sheet is used alone or in combination with a woven or nonwoven fabric. The second sheet may be either elastic or inelastic.

The second sheet may have an air permeability similar to those of breathable films or sheets that are generally used for chemical warmers. The air permeability may, for example, be about 208 to 610 g/m² per day, as measured according to Method A defined in JIS K7129.

A thermoplastic synthetic resin or the like is used as the resin that forms the film or sheet of the second sheet. Specific examples of preferable thermoplastic synthetic resins include polyethylenes, polypropylenes, polyesters, polyamides, polyvinyl alcohols, polyvinyl chloride, polyvinylidene chloride, polyurethanes, polystyrenes, ethylene-vinyl acetate copolymer, polycarbonates, and rubber hydrochloride. These resins are used alone or in combination. Polyethylenes are particularly preferable as the resin that forms the film or sheet.

An oriented film, which is preferably an oriented porous film, or a sheet including the film, is advantageously used as the breathable film or sheet. The oriented porous film, which typically contains an inorganic filler such as calcium carbonate, is made breathable upon formation of pores by drawing. The air permeability of the film can be controlled by controlling the pore size. The breathable film is preferably a laminated olefin-based (particularly a polyethylene-based) oriented and porous film, or a composite sheet thereof with a nonwoven fabric.

The laminated porous film or laminated sheet is typically produced by lamination or other methods. Lamination may be performed according to any known method. For example, lamination may be performed by a method using thermal bonding, or an adhesive such as a hot melt adhesive or an acrylic or urethane adhesive. The surfaces of the laminated film or sheet may be completely bonded, or partially bonded to maintain flexibility.

Examples of nonwoven fabrics that may be laminated to the above-mentioned film or sheet include nonwoven fabrics containing synthetic fibers such as nylon, vinylon, polyesters, rayon, acetate, acrylics, polyethylenes, polypropylenes, and polyvinyl chloride; and nonwoven fabrics containing natural fibers such as cotton, hemp, and silk. The nonwoven fabric has a weight of about 20 to 100 g/m².

1-4. Exothermic Composition

The exothermic composition enclosed in bags may be any composition that generates heat by contact with air. Specifically, in the invention, a composition containing an iron powder, a water-retaining agent, a metal salt, and water is preferably used as the exothermic composition.

The total mass of the iron powder, water-retaining agent, metal salt, and water in the exothermic composition is preferably about 80 to 100 mass %.

The exothermic composition is described in detail below, taking an exothermic composition containing an iron powder, a water-retaining agent, a metal salt, and water as a representative example.

1-4-1. Iron Powder

The heat-generating portion of the invention can exhibit a heating effect when the iron powder generates heat by reacting with oxygen in the air.

Examples of the iron powder include reduced iron and cast iron. These iron powders can be used alone or in combination.

The iron powder may be in the form of granules, fibers, or the like. These forms of iron powders may be used alone or in combination.

The particle size of the granular iron powder is typically about 10 to 300 μm, and preferably about 10 to 100 μm.

The particle size as referred to herein can be determined as follows: One hundred grams of the test specimen (the iron powder or the like) to be measured is placed in an electric vibrating screen including, sequentially from the top, screens of 700 μm, 650 μm, 500 μm, 400 μm, 300 μm, 250 μm, 100 μm, 50 μm, and 10 μm, and vibrated for 15 minutes. The particle size can be subsequently determined by measuring the amount of the test specimen remaining in each screen and the amount of the test specimen passed through each screen.

The amount of the iron powder in the exothermic composition is preferably about 30 to 80 mass %, and more preferably about 45 to 65 mass %.

1-4-2. Water-Retaining Agent

In the invention, the water-retaining agent is a material capable of holding water. Examples of water-retaining agents include porous materials and water-absorbing resins.

Specific examples of porous materials used as the water-retaining agent include activated carbon, wood flour, perlite, and vermiculite.

Activated carbon is capable of incorporating air into micropores on the surface to promote oxygen supply, or of retaining heat so that the heat-release temperature does not vary. Activated carbon has a very porous inside structure, and therefore provides a particularly good water-retaining ability. Furthermore, activated carbon absorbs well not only water, but also water vapor that evaporates upon the generation of heat from the exothermic composition, thereby preventing the water vapor from escaping. Thus, activated carbon can also be very useful as a water-retaining material. Activated carbon can also absorb odor emitted by the oxidation of the iron powder. Activated carbon prepared from, for example, coconut husks, wood, charcoal, coal, and bone black can be preferably used as the activated carbon. The activated carbon may be in the form of granules, fibers, or the like. These forms of activated carbon may be used alone or in combination. Specifically, in the invention, granular activated carbon is preferably used. When granular activated carbon is used, the particle size is preferably about 10 to 300 μm, and more preferably about 10 to 100 μm. The particle size of the activated carbon is measured in the same manner as the particle size of the iron powder.

Wood flour, perlite, and vermiculite may also take any form as long as they can retain water, but are preferably in granular form to improve the feeling of use of the heating device. When wood flour, perlite, or vermiculite in granular form is used, the particle size is typically about 300 μm or less, and preferably about 250 μm or less. The particle size of wood flour, perlite, or vermiculite is also measured in the same manner as the particle size of the iron powder.

Among these porous materials, activated carbon, and vermiculite are preferably used; activated carbon and vermiculite are more preferably used; and activated carbon is particularly preferably used. These porous materials may be used alone or in combination.

Specific examples of water-absorbing resins used as the water-retaining agent include isobutylene-maleic anhydride copolymer, polyvinyl alcohol-acrylic acid copolymer, starch-acrylate graft copolymer, a polyacrylate crosslinked product, acrylate-acrylic ester copolymer, acrylate-acrylamide copolymer, and a crosslinked polyacrylonitrile. Preferable among these water-absorbing resins is a polyacrylate crosslinked product. The particle size of the water-absorbing resin is typically about 100 to 500 μm, and preferably about 250 to 400 μm. The particle size of the water-absorbing resin is also measured in the same manner as the particle size of the iron powder.

These water-absorbing resins can be used alone or in combination.

The porous materials and water-absorbing resins may be used alone or in combination as the water-retaining agent. The water-retaining agent used in the exothermic composition is preferably a porous material, or a combination of a porous material and a water-absorbing resin; more preferably activated carbon, or a combination of activated carbon, a porous material (other than activated carbon), and a water-absorbing resin; and still more preferably a combination of activated carbon, vermiculite, and a polyacrylate crosslinked product.

The amount of the water-retaining agent in the exothermic composition is preferably about 2 to about 30 mass %, and more preferably about 5 to about 20 mass %. More specifically, when a porous material is used alone as the water-retaining agent, the amount of the porous material in the exothermic composition is preferably 10 to 30 mass %, and more preferably about 10 to 20 mass %. When a water-absorbing resin is used alone as the water-retaining agent, the amount of the water-absorbing resin in the exothermic composition is preferably 2 to 10 mass %, and more preferably about 2 to 7 mass %. When a combination of a porous material and a water-absorbing resin is used as the water-retaining agent; the amounts of the porous material and water-absorbing resin in the exothermic composition are preferably 5 to 20 mass % and 1 to 10 mass %, respectively, and more preferably 7 to 20 mass % and 1 to 5 mass %, respectively. Particularly when a combination of activated carbon, a porous material other than activated carbon, and a water-absorbing resin is used as the water-retaining agent, the amounts of the activated carbon, porous material, and water-absorbing resin are preferably 3 to 20 mass %, 1 to 10 mass %, and 1 to 10 mass %, respectively, and more preferably 5 to 15 mass %, 1 to 5 mass %, and 1 to 5 mass %, respectively.

1-4-3. Metal Salt

A metal salt facilitates the oxidation reaction with air, and therefore activates the surface of the iron powder to promote the oxidation reaction of the iron.

A metal salt used in known exothermic compositions may be used as the metal salt. Examples of such metal salts include sulfates such as ferric sulfate, potassium sulfate, sodium sulfate, manganese sulfate, and magnesium sulfate; and chlorides such as cupric chloride, potassium chloride, sodium chloride, calcium chloride, manganese chloride, magnesium chloride, and cuprous chloride. Carbonates, acetates, nitrates, and other salts can also be used. These metal salts can be used alone or in combination.

The particle size of the metal salt is typically about 100 to 700 μm, and preferably about 250 to 650 μm.

The amount of the metal salt in the exothermic composition is preferably about 0.5 to 10 mass %, and more preferably about 1 to 3 mass %.

1-4-4. Water

Usable types of water include distilled water and tap water. The amount of water in the exothermic composition is preferably about 1 to 40 mass %, and more preferably about 20 to 30 mass %.

1-4-5. Mixing the Components

The exothermic composition can be prepared by mixing the above-described components. Mixing may be performed under vacuum or an inert gas atmosphere, as required. Mixing may be performed according to, for example, the method described in U.S. Pat. No. 4,649,895.

1-5. Method for Preparing Heat-Generating Portion

The heat-generating portion is obtained by bonding the first and second sheets so that the exothermic composition is enclosed in one or more sections. When a laminate is used as each of the first and second sheets, the first and second sheets are bonded to each other so that the nonwoven fabric that forms each laminate faces outside (i.e., opposite the face that comes into contact with the enclosed exothermic composition). At this moment, in order to form each section that encloses the exothermic composition, all of the regions of the heat-generating portion excluding each section are bonded. For example, referring to each of FIGS. 1 to 3, the first sheet and second sheet are bonded to each other in the regions of the heat-generating portion 1 except for the sections 2.

As shown in FIG. 3, the heat-generating portion may have a through-hole 5 between adjacent sections. For example, when the heating device of the invention is worn on the knee, the presence of the through-hole 5 allows the kneecap to enter the through-hole 5, thereby enhancing the conformability of the heating device to the knee joint. The heating device may also optionally have through-hole(s) for use on a joint other than the knee joint.

Although FIG. 3 illustrates the through-hole 5, the heating device may not have a through-hole. Further, although each of FIGS. 1 and 2 does not illustrate a through-hole, the heating device may have a through-hole between adjacent sections. The number of through-holes is typically one, but may also be two or more.

Examples of usable bonding methods include, but are not limited to, thermal bonding and methods of bonding using resin components as mentioned above.

2. Band

The band portion is formed of an extensible material, and attached to the heat-generating portion. By winding the band portion around the body, the heat-generating portion can be held onto the body (an affected area for a patient), allowing the heat-generating portion to be kept in contact with the body. As shown in FIG. 1, in the heating device of the invention, the heat-generating portion 1 is connected to one end of the band portion 3. In FIG. 1, the band portion 3 is connected to only one end of the heat-generating portion 1; however, as shown in FIG. 2, the band portion 3 may also be connected to opposite ends of the heat-generating portion 1. Further, as shown in FIG. 3, two or more band portions may be connected to one end of the heat-generating portion. That is, a pair of band portions may be connected in parallel at a distance from each other to one end of the heat-generating portion.

Examples of connecting methods include, but are not limited to, bonding with a known adhesive, fixing with a thread, ultrasonic welding, and other methods. Moreover, as shown in each of FIGS. 1 to 3, the other end of the band portion 3 is typically provided with a bonded portion 4 that provides excellent adhesion with the heat-generating portion and/or the band portion, in order to keep the heat-generating portion in contact with the body. The bonded portion 4 may, for example, be MAGICTAPE (registered trademark).

The size of the band portion is not limited, and may be suitably adjusted according to the size of the area to which the heating device is applied.

For example, when the heating device is applied to the wrist or ankle, it is preferable that the band portion have a long side of about 5 to 32 cm, and a short side of about 3 to 20 cm; and it is more preferable that the band portion have a long side of about 10 to 20 cm, and a short side of about 5 to 12 cm.

When the heating device is applied to the knee, it is preferable that the band portion have a long side of about 10 to 50 cm, and a short side of about 5 to 20 cm; and it is more preferable that the band portion have a long side of about 20 to 30 cm, and a short side of about 10 to 17 cm.

When the heating device is applied to the waist, it is preferable that the band portion have a long side of about 10 to 50 cm, and a short side of about 5 to 20 cm; and it is more preferable that the band portion have a long side of about 20 to 30 cm, and a short side of about 10 to 17 cm.

When the heating device is applied to the neck, it is preferable that the band portion have a long side of about 10 to 50 cm, and a short side of about 5 to 20 cm; and it is more preferable that the band portion have a long side of about 20 to 30 cm, and a short side of about 10 to 17 cm.

When the heating device is applied to the elbow, it is preferable that the band portion have a long side of about 10 to 50 cm, and a short side of about 5 to 20 cm; and it is more preferable that the band portion have a long side of about 20 to 30 cm, and a short side of about 10 to 17 cm.

When the heating device is applied to a finger, it is preferable that the band portion have a long side of about 3 to 15 cm, and a short side of about 3 to 10 cm; and it is more preferable that the band portion have a long side of about 3 to 10 cm, and a short side of about 3 to 5 cm.

When the long side and short side of the band portion are within these ranges, the heat-generating portion can be advantageously brought into contact with the body.

The elongation of the band portion is not limited as long as it is within a range such that the band portion can surround the heat-generating portion to advantageously keep the heat-generating portion in contact with the body. If the extensibility of the band portion is too low, the heat-generating portion does not fit along a joint. Therefore, the extensibility is, for example, such that the force required to elongate the band portion 150% is typically 115 N or less, preferably 0.3 to 115 N, and more preferably 0.3 to 102 N.

The force required to elongate the band portion 150% is herein a value determined by cutting the band portion to 100×50 mm, and measuring the force (N) required to elongate the band portion 150% when pulling the band portion in the longitudinal direction, using a tensile testing machine (AGS-H Shimadzu Corporation).

The specific structure of the band portion is not limited as long as the band portion has a certain degree of extensibility. The band portion is preferably made of a nonwoven or woven fabric of a natural or synthetic fiber, and more preferably has multiple vent holes. The thickness of the band portion is typically about 0.1 to 1 mm, and preferably about 0.5 to 1 mm.

An example of a commercially available band portion is “Optiflex”, produced by Golden Phoenix Fiberwebs Inc.

The air permeability of the band portion is determined by the pore size of the vent holes. The air permeability can be adjusted by, for example, providing the band portion with minute holes using a needle, a laser, electric discharge machining, or the like.

The total area of the vent holes in the band portion is preferably about 3 to 8%, and more preferably about 4 to 6%, of the area of the band portion. The value of the total area can be determined as follows: The number of vent holes in a 50×50 mm portion of the band portion is visually counted, the counted number is multiplied by the area of each hole (a radius of 0.5 mm), and resulting value is divided by 2,500 m².

3. Embodiments of Use of Heating Device

The heat-generating portion (the exothermic composition) that forms the heating device of the invention generates heat in the presence of air. Therefore, the heating device is typically distributed while being enclosed in an airtight package.

A feature of the heating device of the invention is that the heat-generating portion is directly contacted with the skin. Moreover, as stated above, the heating device of the invention has a heat-generating portion and a band portion connected thereto, which are integrally formed with each other. The heating device of the invention allows the heat-generating portion to come into contact with the body, thereby imparting a heating effect to the body. Because the heat-generating portion is fixed with the band portion to the area to which the heating device is applied, the heating device of the invention is not easily shifted from the affected area (the worn position), even during initial use. The heating device of the invention can typically maintain the temperature at about 38 to 42° C.

The term “heating effect” as used herein means warming the affected area by generating heat. The heating effect can, for example, improve the blood circulation of the affected area, and promote the restoration of the affected area while removing waste products from the tissue of the affected area. The heating effect allows the affected area to be warmed to about 38 to 42° C., and preferably about 39 to 41° C.

As shown in, for example, FIG. 4, the heat-generating portion is fixed by winding the band portion around the body.

Therefore, the heating device of the invention can ameliorate diseases and symptoms of the wrist, ankle, knee, neck, elbow, fingers, waist, and the like. Furthermore, the heating device of the invention can be advantageously used as a device for rehabilitation after treatment. Because of its excellent heating effect, the heating device of the invention can be advantageously used not only for treatment and rehabilitation, but also as a chemical warmer for warming the body.

Furthermore, in the heating device of the invention, the weight per unit area of the exothermic composition accommodated in each section and the area of each section are within the above-defined ranges. For this reason, during initial use, the heat-generating portion is flexible, and thus covers the area of the body to which the heating device is applied (for example, a wrist joint, ankle joint, knee joint, neck, elbow, finger joint, or waist) while it fits along the area of the body. The heating device thus provides good feeling of use, and effectively promotes the blood circulation of the area to which it is applied.

Furthermore, in the invention, the weight of accommodated exothermic composition per unit area of each section and the area of each section are within the above-defined ranges. For this reason, during later use, the heat-generating portion hardens while it fits along the shape of the area to which the heating device is applied. Restricting the movement of the area to which the heating device is applied produces, in addition to the heating effect, the effect of protecting the area (an effect similar to wearing a plaster cast) by fixing the area. This promotes the prevention and treatment of joint pain or the like.

According to the invention, the heating device is advantageously used as a heating or therapeutic device for the treatment of joint pain or the like, by covering a wrist joint, ankle joint, knee joint, neck, elbow, finger joint, waist, or the like. That is, joint pain, lumbago, and the like can be treated using the heating device of the invention (a treatment method). In particular, during later use, the heat-generating portion hardens along the shape of a joint or the like. Therefore, the heating device can be advantageously used as a heating or therapeutic device for the treatment of joint pain or the like, by covering a wrist joint, ankle joint, knee joint, neck, elbow, finger joint, or waist, and by fixing the joint or the like with the heat-generating portion that has hardened by the oxidation of the exothermic composition. Thus, joint pain, lumbago, and the like can be treated using the heating device of the invention (a treatment method).

### Advantageous Effects of Invention

In the invention, by setting the weight per unit area of the exothermic composition in each section that accommodates the exothermic composition within the above-defined range, and by setting the area of each section that accommodates the exothermic composition within the above-defined range, it is possible to increase the flexibility of the heating device during initial use, allowing the heat-generating portion to fit along the area of the body to which the heating device is applied; and effectively immobilize the area to which the heating device is applied during later use by the hardening of the heat-generating portion. This enhances the prevention and treatment of, for example, joint pain, lumbago, and the like. According to the invention, there is provided a heating device highly capable of alleviating pains such as in a wrist joint, ankle joint, knee joint, neck, elbow, finger joint, waist, and the like, and of speeding the pace of recovery from such pains.

### BRIEF DESCRIPTION OF DRAWINGS

FIG. 1 is a schematic diagram of the heating device of the invention.

FIG. 2 is a schematic diagram of the heating device of the invention.

FIG. 3 is a schematic diagram of the heating device of the invention.

FIG. 4 is a diagram showing an embodiment of the use of the heating device of the invention.

### REFERENCE SIGNS LIST

1. Heat-Generating Portion
2. Section
3. Band Portion
4. Bonded Portion
5. Through-Hole

### DESCRIPTION OF EMBODIMENTS

The present invention is described in detail with reference to the following Examples, Comparative Examples, and Test Examples; however, the invention is not limited to these examples.

### Examples and Comparative Examples

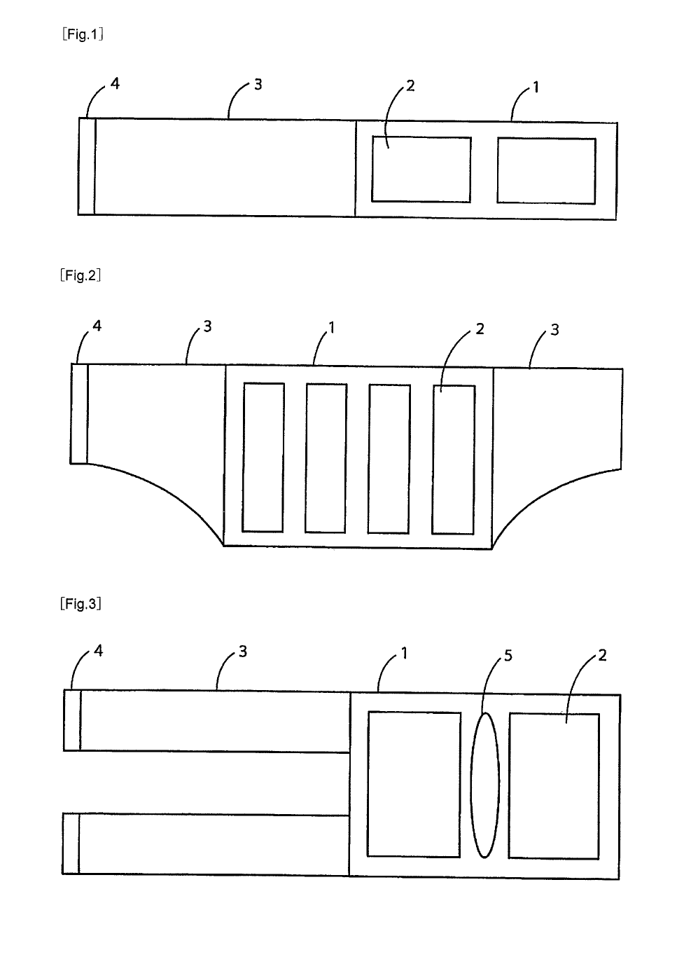

Heating devices having the structures of FIGS. 1, 2, and 3 were prepared for evaluations on the wrist, waist, and knee, respectively.

Heat-Generating Portion

1) Exothermic Composition

An exothermic composition was prepared by mixing an iron powder with a particle size of 50 μm, activated carbon with a particle size of 200 μm, salt with a particle size of 400 μm, water, vermiculite with a particle size of 100 μm, and an acrylic acid polymer partial sodium salt crosslinked product with a particle size of 400 μm. The amounts of the iron powder, activated carbon, sodium chloride, water, vermiculite, and sodium polyacrylate in the exothermic composition were 55 mass %, 13 mass %, 1 mass %, 26 mass %, 3 mass %, and 2 mass %, respectively.

2) First Sheet

A nonwoven fabric (weight: 30 g/m²) produced by spunlace using polyethylene terephthalate was laminated to a film containing polyethylene as a resin component, thereby preparing a laminate with a length of 31.5 cm and a width of 5 m.

3) Second Sheet

A laminate of a PET-SL nonwoven fabric/LDPE was prepared (air permeability: 300 g/m² per day). A fiber sheet (weight: 30 g/m²), which was made into a composite fiber by thermal bonding using polypropylene and polyethylene, was laminated to a porous film (thickness: 70 μm), which was made porous by drawing a film principally containing an olefin-based resin and an inorganic filler (calcium carbonate), thereby preparing a laminate with a length of 31.5 cm and a width of 5 m.

4) Preparation of Heat-Generating Portion

Immediately after the preparation of the exothermic composition, the exothermic composition was sealed using the first sheet and second sheet. In this way, rectangular heat-generating portions each for the knee (with a long side of 36 cm and a short side of 20 cm) and for the waist (with a long side of 36 cm and a short side of 20 cm) were prepared.

Specifically, each heat-generating portion was prepared by forming two rectangular sections that enclosed the exothermic composition, and bonding all of the regions of the heat-generating portion except for the sections on the first and second sheets, in such a manner that the polyethylene resin film of the first sheet and the polyethylene resin film of the second sheet came into contact with each other. Ten types of heat-generating portions having areas of each section of 9 cm², 15 cm², 24 cm², 32 cm², 45 cm², 55 cm², 80 cm², 117 cm², 150 cm², and 180 cm² were prepared.

The length (the short side of the heat-generating portion) and the width (the long side of the heat-generating portion) of each section of each of these heat-generating portions were as follows: 3 cm long and 3 cm wide (an area of 9 cm²); 5 cm long and 3 cm wide (an area of 15 cm²); 6 cm long and 4 cm wide (an area of 24 cm²); 4 cm long and 8 cm wide (an area of 32 cm²); 9 cm long and 5 cm wide (an area of 45 cm²); 11 cm long and 5 cm wide (an area of 55 cm²); 10 cm long and 8 cm wide (an area of 80 cm²); 13 cm long and 9 cm wide (an area of 117 cm²); 15 cm long and 10 cm wide (an area of 150 cm²); and 18 cm long and 10 cm wide (an area of 180 cm²). Bonding of each first sheet and each second sheet was performed at 130° C. by thermal bonding.

Further, for each of the heat-generating portions having 10 different areas of each section, the weight per unit area of the exothermic composition in each section was varied to the following 15 types: 0.08 g/cm²; 0.11 g/cm²; 0.15 g/cm²; 0.2 g/cm²; 0.33 g/cm²; 0.4 g/cm²; 0.54 g/cm²; 0.57 g/cm²; 0.6 g/cm²; 0.67 g/cm²; 0.71 g/cm²; 0.74 g/cm²; 0.88 g/cm²; 0.94 g/cm²; and 0.96 g/cm². Accordingly, a total of 150 types of heat-generating portions were prepared. Note that the weight per unit area was measured according to the method described above.

Band

For Evaluation on the Knee

Bands were prepared by providing a band portion 13 cm long, 22 cm wide, and 0.8 mm thick (tradename “Optiflex”; Golden Phoenix Fiberwebs Inc.) with uniform perforations at regular intervals using a 0.5 mm-wide drill. Each band portion was provided with MAGICTAPE (registered trademark) 10 cm long and 2.5 cm wide.

For Evaluation on the Wrist

Bands 8 cm long, 20 cm wide, and 0.8 mm thick (tradename “Optiflex”; Golden Phoenix Fiberwebs Inc.) were used as the band portions.

Each band portion was bonded to one end of each heat-generating portion using an ultrasonic welding machine. The band portion was provided with MAGICTAPE (registered trademark) 7 cm long and 2.5 cm wide.

For Evaluation on the Waist

Bands 10 cm long, 20 cm wide, and 0.8 mm thick (tradename “Optiflex”; Golden Phoenix Fiberwebs Inc.) were used as the band portions.

Bands were bonded to both ends of each heat-generating portion using an ultrasonic welding machine. One of the band portions was provided with MAGICTAPE (registered trademark) 8 cm long and 2.5 cm wide.

Heating Device

Heating devices were prepared by ultrasonically fixing each band portion to end(s) of each heat-generating portion. Each of the heating devices prepared in the Examples and Comparative Examples was sealed in a bag made of a polyvinylidene chloride-coated film (KOP), in order to prevent contact with air.

Test Examples 1 to 7 below were conducted immediately after the removal of heating devices from the bag of the polyvinylidene chloride-coated film.

### Test Example 1

### Evaluation of Flexibility During Initial Use

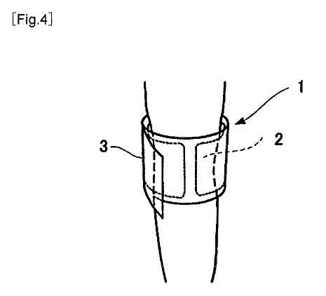

Ten healthy subjects wore each of the heating devices prepared in the Examples and Comparative Examples, having different areas of each section and different weights per unit area of the exothermic composition, as shown in Table 1 below. The flexibility of each heating device was evaluated immediately after the beginning of the use of the heating device. Specifically, as shown in FIG. 4, in order to keep the first sheet-side of the heat-generating portion in contact with the knee joint, the knee was surrounded with the heat-generating portion and band portion. The heat-generating portion was then fixed with the MAGICTAPE (registered trademark) of the band portion. The ratings used in Table 1 are defined as follows:

Ratings

5: Excellent flexibility and fit

4: Good flexibility and fit

3: Normal flexibility and fit

2: Somewhat poor flexibility and fit

1: Poor flexibility and fit

A: An average rating of 4.5 or more, based on the ratings of the 10 subjects.

B: An average rating of 4.0 or more and less than 4.5, based on the ratings of the 10 subjects.

C: An average rating of 3.5 or more and less than 4.0, based on the ratings of the 10 subjects.

D: An average rating of less than 3.0, based on the ratings of the 10 subjects.

TABLE 1
1. Evaluation of Flexibility during Initial Use

| g/cm² | | | | | | | | | | |
|---|---|---|---|---|---|---|---|---|---|---|
| 0.96 | D | D | D | D | D | D | D | D | D | D |
| 0.94 | D | C | C | C | C | C | C | C | C | D |
| 0.88 | D | C | C | C | C | C | C | C | C | D |
| 0.74 | D | C | C | C | C | C | C | C | C | D |
| 0.71 | D | C | B | B | B | B | B | B | C | D |
| 0.67 | D | C | B | B | B | B | B | B | C | D |
| 0.6 | D | C | B | A | A | A | A | B | C | D |
| 0.57 | D | C | B | A | A | A | A | B | C | D |
| 0.54 | D | C | B | A | A | A | A | B | C | D |
| 0.4 | D | C | B | A | A | A | A | B | C | D |
| 0.33 | D | C | B | A | A | A | A | B | C | D |
| 0.2 | D | C | B | A | A | A | A | B | C | D |
| 0.15 | D | C | B | B | B | B | B | B | C | D |
| 0.11 | D | C | C | C | C | C | C | C | C | D |
| 0.08 | D | C | C | C | C | C | C | C | C | D |
| | 9 | 15 | 24 | 32 | 45 | 56 | 80 | 117 | 150 | 180 |
| | | | | | | | | | | cm² |

The results of Test Example 1 show that good flexibility during initial use was obtained when the weight per unit area of the exothermic composition was 0.08 to 0.94 g/cm². Particularly when the weight per unit area of the exothermic composition was 0.15 to 0.71 g/cm², excellent flexibility during initial use was obtained. Further, the results show that when the area of each section was too small, i.e., 9 cm², the heat-generating portions did not flexibly conform to the joint. Conversely, when the area of each section was too large, i.e., 180 cm², the heat-generating portions could not conform to the joint. The results A and B indicate that the heating devices conformed to the shape of the knee joint of each individual, and the heat-generating portions had neatly hardened after use. Similar tests were also conducted for the wrist and waist. The results were the same as the results obtained for the knee joint.

Test Example 2

### Evaluation of the Degree of Hardening of the Heat-Generating Portion during Later Use

Ten healthy subjects wore each of the heating devices prepared in the Examples and Comparative Examples, having different areas of each section and different weights per unit area of the exothermic composition, as shown in Table 2 below. The degree of hardening of the heat-generating portion of each heating device during later use was evaluated. Specifically, the degree of hardening of each heat-generating portion from the beginning of wearing the heating device on the knee to the completion of the generation of heat in Test Example 1 was evaluated. The ratings used in Table 2 are defined as follows:

Ratings:

5: The heat-generating portion showed a very high degree of hardening.

4: The heat-generating portion showed a high degree of hardening.

3: The heat-generating portion showed a normal degree of hardening.

2: The heat-generating portion showed a somewhat low degree of hardening.

1: The heat-generating portion showed a low degree of hardening.

A: An average rating of 4.5 or more, based on the ratings of the 10 subjects.

B: An average rating of 4.0 or more and less than 4.5, based on the ratings of the 10 subjects.

C: An average rating of 3.5 or more and less than 4.0, based on the ratings of the 10 subjects.

D: An average rating of less than 3.0, based on the ratings of the 10 subjects.

TABLE 2
2. Evaluation of the Degree of Hardening of the Heat-
Generating Portion during Later Use

| g/cm² | | | | | | | | | | |
|---|---|---|---|---|---|---|---|---|---|---|
| 0.96 | A | A | A | A | A | A | A | A | A | A |
| 0.94 | A | A | A | A | A | A | A | A | A | A |
| 0.88 | A | A | A | A | A | A | A | A | A | A |
| 0.74 | A | A | A | A | A | A | A | A | A | A |
| 0.71 | A | A | A | A | A | A | A | A | A | A |
| 0.67 | A | A | A | A | A | A | A | A | A | A |
| 0.6 | A | A | A | A | A | A | A | A | A | A |
| 0.57 | A | A | A | A | A | A | A | A | A | A |
| 0.54 | A | A | A | A | A | A | A | A | A | A |
| 0.4 | A | A | A | A | A | A | A | A | A | A |
| 0.33 | A | A | A | A | A | A | A | A | A | A |
| 0.2 | A | A | A | A | A | A | A | A | A | A |
| 0.15 | B | B | B | B | B | B | B | B | B | B |
| 0.11 | C | C | C | C | C | C | C | C | C | C |
| 0.08 | D | D | D | D | D | D | D | D | D | D |
| | 9 | 15 | 24 | 32 | 45 | 56 | 80 | 117 | 150 | 180 |
| | | | | | | | | | | cm² |

The results of Test Example 2 show that a high strength during later use was obtained when the weight per unit area of the exothermic composition was 0.11 g/cm² or more, irrespective of the area of each section. Similar tests were also conducted for the wrist and waist. The results were the same as the results obtained for the knee joint.

### Summary of Test Examples 1 and 2

Summarizing the results of Test Examples 1 and 2, Table 3 below shows the heating devices that provided satisfactory flexibility and fit during initial use, as well as satisfactory strength during later use. The ratings used in Table 3 are the same as those used in Test Examples 1 and 2.

TABLE 3
Summary of Test Examples 1 and 2

| g/cm² | | | | | | | | | | |
|---|---|---|---|---|---|---|---|---|---|---|
| 0.96 | D | D | D | D | D | D | D | D | D | D |
| 0.94 | D | C | C | C | C | C | C | C | C | D |
| 0.88 | D | C | C | C | C | C | C | C | C | D |
| 0.74 | D | C | C | C | C | C | C | C | C | D |
| 0.71 | D | C | B | B | B | B | B | B | C | D |
| 0.67 | D | C | B | B | B | B | B | B | C | D |
| 0.6 | D | C | B | A | A | A | A | B | C | D |
| 0.57 | D | C | B | A | A | A | A | B | C | D |
| 0.54 | D | C | B | A | A | A | A | B | C | D |
| 0.4 | D | C | B | A | A | A | A | B | C | D |
| 0.33 | D | C | B | A | A | A | A | B | C | D |
| 0.2 | D | C | B | A | A | A | A | B | C | D |
| 0.15 | D | C | B | B | B | B | B | B | C | D |
| 0.11 | D | C | C | C | C | C | C | C | C | D |
| 0.08 | D | D | D | D | D | D | D | D | D | D |
| | 9 | 15 | 24 | 32 | 45 | 56 | 80 | 117 | 150 | 180 |
| | | | | | | | | | | cm² |

The results of Test Examples 1 and 2 show that, when the area of each section in which the exothermic composition is enclosed is 15 to 150 cm², and the weight per unit area of the exothermic composition in each section is 0.11 to 0.94 g/cm², heating devices having good flexibility during initial use and high strength of heat-generating portion during later use can be obtained. The results also show that, when the area of each section is 24 to 117 cm², and the weight per unit area of the exothermic composition is 0.15 to 0.71 g/cm², heating devices having better flexibility during initial use and higher strength of heat-generating portion during later use can be obtained. Similar tests were also conducted for the wrist and waist. The results were the same as the results obtained for the knee joint.

### Test Example 3

### Relationship between the Area of Each Section and the Weight per Unit Area of the Exothermic Composition, and the Pain Alleviation and Pace of Recovery

Each of the heating devices shown in Table 4 below was worn by five patients suffering from pain in the knee, and the relationship between the area of each section and the weight per unit area of the exothermic composition, and the pain alleviation and the pace of recovery from the pain was evaluated. The ratings used in Table 4 are defined as below. The test results were obtained by making each patient wear one sample of each heating device for 24 hours from the beginning of the generation of heat, and repeating this for five successive days. The heating time of one sample was 12 hours.

Ratings

Degree of Knee Pain Alleviation

5: A very high degree of knee pain alleviation

4: A high degree of knee pain alleviation

3: A normal degree of knee pain alleviation

2: A somewhat low degree of knee pain alleviation

1: A low degree of knee pain alleviation

Level of Satisfaction with Time Needed for Pain Alleviation

5: An average of less than 2.0 days

4: An average of 2.0 days or more and less than 2.5 days

3: An average of 2.5 days or more and less than 3.0 days

2: An average of 3.0 days or more and less than 3.5 days

1: An average of 3.5 days or more and less than 4.0 days

A: An average rating of 4.5 or more, based on the ratings of the five subjects.

B: An average rating of 4.0 or more and less than 4.5, based on the ratings of the five subjects.

C: An average rating of 3.5 or more and less than 4.0, based on the ratings of the five subjects.

D: An average rating of less than 3.0, based on the ratings of the five subjects.

TABLE 4
Pain Alleviation and Pace of Recovery

| | | | | | | | | | | Comparative | | |
|---|---|---|---|---|---|---|---|---|---|---|---|---|
| | Examples | | | | | | | | | Examples | | |
| | 1 | 2 | 3 | 4 | 5 | 6 | 7 | 8 | 9 | 1 | 2 | 3 |
| Weight per | 0.4 | 0.3 | 0.6 | 0.7 | 0.7 | 0.2 | 0.4 | 0.3 | 0.11 | 0.6 | 1 | 0.4 |
| Unit Area of | | | | | | | | | | | | |
| Exothermic | | | | | | | | | | | | |
| Composition | | | | | | | | | | | | |
| (g/cm²) | | | | | | | | | | | | |
| Area of Each | 56 | 80 | 32 | 56 | 117 | 24 | 15 | 150 | 56 | 9 | 56 | 180 |
| Section (cm²) | | | | | | | | | | | | |
| Pain | A | A | A | B | B | B | C | C | C | D | D | D |
| Alleviation | | | | | | | | | | | | |
| Pace of | A | A | A | B | B | B | C | C | C | D | D | D |
| Recovery | | | | | | | | | | | | |

The results of Test Example 3 show that a heat-generating portion requires both an appropriate degree of flexibility during initial use and strength during later use, in order to enhance the alleviation of joint pain (the degree of knee pain alleviation) and the pace of recovery (the level of satisfaction with the time needed for pain alleviation). Further, similar tests were also conducted for the wrist and waist. The results were the same as the results obtained for the knee joint.

### Test Example 4

### Comparison between the Heating Device of the Invention and a Heating Device with Multiple Small Sections

Five patients suffering from pain in the knee wore a heating device of the invention having the weight per unit area of the exothermic composition and the area of each section of Example 1 (the weight per unit area of the exothermic composition: 0.4 g/cm², the area of each section: 56 cm²), and a heating device of Comparative Example 4 having multiple small sections on the heat-generating portion in its body portion, and having an appropriate degree of flexibility. Various comparisons were made between these heating devices. The heating device of Comparative Example 4 was structurally identical to that of Example 1, except that the area of each section in the heat-generating portion was 7.85 cm², the weight per unit area of the exothermic composition was 0.38 g/cm², and there were 12 sections on the heat-generating portion in its body portion. The test results were obtained by making each patient wear one sample of each heating device for 24 hours from the beginning of the generation of heat, and repeating this for five successive days.

Degree of Knee Pain Alleviation

5: A very high degree of knee pain alleviation

4: A high degree of knee pain alleviation

3: A normal degree of knee pain alleviation

2: A somewhat low degree of knee pain alleviation

1: A low degree of knee pain alleviation

Sensation of Warmth

5: Excellent heat transfer from the heat-generating portion to the knee

4: Good heat transfer from the heat-generating portion to the knee

3: Normal heat transfer from the heat-generating portion to the knee

2: Somewhat poor heat transfer from the heat-generating portion to the knee

1: Poor heat transfer from the heat-generating portion to the knee

Flexibility and Fit

5: Excellent flexibility and Fit

4: Good flexibility and fit

3: Normal flexibility and fit

2: Somewhat poor flexibility and fit

1: Poor flexibility and fit

Level of Satisfaction with Time Needed for Pain Alleviation

5: An average of less than 2.0 days

4: An average of 2.0 days or more and less than 2.5 days

3: An average of 2.5 days or more and less than 3.0 days

2: An average of 3.0 days or more and less than 3.5 days

1: An average of 3.5 days or more and less than 4.0 days

A: An average rating of 4.5 or more, based on the ratings of the five subjects.

B: An average rating of 4.0 or more and less than 4.5, based on the ratings of the five subjects.

C: An average rating of 3.5 or more and less than 4.0, based on the ratings of the five subjects.

D: An average rating of less than 3.0, based on the ratings of the five subjects.

TABLE 5

| | | | | Level of | Average |
|---|---|---|---|---|---|
| | | | | Satisfaction | Number of |
| | Degree of | | | with Time Needed | Days Needed |
| | Knee Pain | Sensation | Flexibility | for Pain | for Pain |
| | Alleviation | of Warmth | And Fit | Alleviation | Alleviation |
| Example 1 | A | A | B | A | 1.8 |
| Comparative | B | A | A | D | 2.8 |
| Example 4 | | | | | |

The results of Test Example 4 show that the heating device of Comparative Example 4 in which each single section was small had high flexibility; however, because each single section of the heat-generating portion was small, the single section of the heat-generating portion could not widely cover the joint. For this reason, even though the strength of the heat-generating portion increased during later use, the heating device could not fix the joint. Accordingly, when the heating device of Comparative Example 4 was used, a longer period of time was required until the pain was relieved, and the level of satisfaction of the patients with knee pain was low.

In contrast, it can be seen that, in the heating device of Example 1, the heat-generating portion hardened while covering the knee joint during later use, so that the heating device greatly contributed to speeding the pace of recovery. It is believed that the hardened heat-generating portion functioned as a plaster cast, thereby reinforcing the weakened ligaments.

Test Example 5

### Comparison between the Heating Device of the Invention and a Heating Device to which an Adhesive Material was Applied

Five subjects suffering from lumbago wore each of a heating device with an adhesive portion obtained as in Example 1, except that the band portion was removed and an acrylic adhesive material was applied to the entire surface of the polyethylene film of the heat-generating portion that is directly contacted with the skin; and the heating device of Example 1 (the weight per unit area of the exothermic composition: 0.33 g/cm², the area of each section: 56 cm²). These heating devices were compared in terms of the degree of lumbago alleviation, the sensation of warmth, as well as the flexibility and fit. The ratings used are the same as those defined in Test Example 4. The test results were obtained by making each patient wear one sample of each heating device for 24 hours from the beginning of the generation of heat.

TABLE 6

| | Degree of Lumbago | Sensation | Flexibility |
|---|---|---|---|
| | Alleviation | of Warmth | And Fit |
| Example 1 | A | A | B |
| with Adhesive | D | B | D |
| Material | | | |

The results of Test Example 5 show that, with the heating device to which the adhesive material was applied, the skin in contact with the adhesive portion was pulled; therefore, the heating device had significantly poor flexibility and fit as compared to the heating device of Example 1. Furthermore, when the adhesive material was used, the heat-generating portion could not conform to the curve of the affected area, and thus did not have flexibility during initial use. Therefore, the heating device had a low degree of lumbago alleviation.

Test Example 6

### Comparison between the Heating Device of the Invention and a Heating Device having a Heat-Generating Portion Accommodated in a Supporter

Five patients suffering from pain in the knee wore each of the heating device of Example 1 (the weight per unit area of the exothermic composition: 0.4 g/cm², the area of each section: 56 cm²) and a heating device as taught in Patent Literature 2 having a heat-generating portion (a chemical warmer) accommodated in a supporter (“Hiza Hotton” [“Knee Warmer”]; Kiribai Chemical Co., Ltd.). These heating devices were compared in terms of the degree of knee pain alleviation, the sensation of warmth, as well as the flexibility and fit. The ratings used are the same as those defined in Test Example 4. The test results were obtained by making each patient wear one sample of each heating device for 24 hours from the beginning of the generation of heat.

TABLE 7

| | Degree of Lumbago | Sensation | Flexibility |
|---|---|---|---|
| | Alleviation | of Warmth | And Fit |
| Example 1 | A | A | B |
| Supporter | D | D | A |

The results of Test Example 6 show that, when the heating device having a heat-generating portion (a chemical warmer) accommodated in a supporter was used, the supporter compressed the area to which the heating device was applied, to thereby conform to the curve, allowing the heat-generating portion to flexibly fit along the area. However, because the chemical warmer was inserted into the supporter, it was not directly contacted with the skin, resulting in a poor sensation of warmth. Furthermore, the chemical warmer inserted into bags for accommodating the chemical warmer in the supporter moved in the bags of the supporter. Therefore, the heat-generating portion did not harden along the shape of the knee joint during later use, and poorly fixed the knee joint. The heating device thus had a low degree of knee pain alleviation.

### Test Example 7

### Relationship between the Compressive Force and the Force Required for the Band to Exhibit an Elongation of 150%

Bands having various elongations were each cut to 100×50 mm. Each of the cut band portions was pulled in the longitudinal direction, using a tensile testing machine (AGS-H Shimadzu Corporation), and the force (N) required to elongate the band portion 150% was measured. Each of the band portions having various elongations was combined with a heat-generating portion having the weight per unit area of the exothermic composition and the area of each section of Example 1. Each of the thus-prepared heating devices was worn by five healthy subjects, and evaluated in terms of the flexibility and fit on the knee, the degree of knee pain alleviation, the sensation of warmth, and the level of satisfaction with the time needed for pain alleviation. The ratings used are the same as those defined in Test Example 4. The test results were obtained by making each subject wear one sample of each heating device for 24 hours from the beginning of the generation of heat, and repeating this for five successive days.

TABLE 8

| | 0.3 | 1.5 | 5 | 27 | 45 | 55 | 63 | 85 | 102 | 115 | 123 |
|---|---|---|---|---|---|---|---|---|---|---|---|
| Flexibility And Fit | A | A | A | A | A | A | A | A | A | B | D |
| Degree of Knee Pain Alleviation | A | A | A | A | A | A | A | A | A | B | D |
| Sensation of Warmth | A | A | A | A | A | A | A | A | A | A | A |
| Level of Satisfaction with | A | A | A | A | A | A | A | A | A | B | D |
| Time Needed for Pain Alleviation | | | | | | | | | | | |

The results of Test Example 7 show that, although the band portion requiring a force of 123 N to exhibit an elongation of 150% was capable of fixing the knee by compression, the lack of extensibility resulted in poor flexibility of the heat-generating portion, and poor fit on the joint. Conversely, the band portions requiring a force of 115 N or less to exhibit an elongation of 150% had excellent flexibility of the heat-generating portion and excellent fit on the joint, and also had an excellent sensation of warmth, a very high degree of knee pain alleviation, and a very high level of satisfaction with the time needed for pain alleviation.

1. A heating device attached to a body, comprising:
a heat-generating portion having an exothermic composition enclosed in one or more sections, the exothermic composition generating heat by contact with air; and
at least one band portion formed of an extensible material and attached to the heat-generating portion;
the exothermic composition accommodated in each section having a weight per unit area of 0.11 to 0.94 g/cm²; and
each section having an area of 15 to 150 cm².

2. The heating device according to claim 1, wherein the number of the one or more sections of the heat-generating portion is one, or two to four per area to which the heating device is applied, and the two to four sections are disposed adjacent to one another.

3. The heating device according to claim 1, wherein the exothermic composition accommodated in each section has a weight per unit area of 0.15 to 0.71 g/cm², and each section has an area of 24 to 117 cm².

4. The heating device according to claim 1, wherein the band portion has extensibility such that a force required to elongate the band portion 150% is 115 N or less.

5. The heating device according to claim 1, which is applied to any of a wrist joint, ankle joint, knee joint, neck, elbow, finger joint, or waist.

6. The heating device according to claim 1, which is used for the treatment of joint pain or lumbago.

7. The heating device according to claim 1, which is used for the treatment of joint pain or lumbago by covering a wrist joint, ankle joint, knee joint, neck, elbow, finger joint, or waist.

8. A method for treating joint pain or lumbago comprising covering a wrist joint, ankle joint, knee joint, neck, elbow, finger joint, or waist with the heating device of claim 1, and immobilizing the joint or waist with the heat-generating portion that has hardened by oxidation of the exothermic composition.

9. Use of the heating device of claim 1, for manufacturing a device for treating joint pain or lumbago.

10. The heating device according to claim 2, wherein the exothermic composition accommodated in each section has a weight per unit area of 0.15 to 0.71 g/cm², and each section has an area of 24 to 117 cm².

11. The heating device according to claim 2, wherein the band portion has extensibility such that a force required to elongate the band portion 150% is 115 N or less.

12. The heating device according to claim 3, wherein the band portion has extensibility such that a force required to elongate the band portion 150% is 115 N or less.

13. The heating device according to claim 2, which is applied to any of a wrist joint, ankle joint, knee joint, neck, elbow, finger joint, or waist.

14. The heating device according to claim 3, which is applied to any of a wrist joint, ankle joint, knee joint, neck, elbow, finger joint, or waist.

15. The heating device according to claim 2, which is used for the treatment of joint pain or lumbago.

16. The heating device according to claim 3, which is used for the treatment of joint pain or lumbago.

17. The heating device according to claim 2, which is used for the treatment of joint pain or lumbago by covering a wrist joint, ankle joint, knee joint, neck, elbow, finger joint, or waist.

18. The heating device according to claim 3, which is used for the treatment of joint pain or lumbago by covering a wrist joint, ankle joint, knee joint, neck, elbow, finger joint, or waist.

19. A method for treating joint pain or lumbago comprising covering a wrist joint, ankle joint, knee joint, neck, elbow, finger joint, or waist with the heating device of claim 2, and immobilizing the joint or waist with the heat-generating portion that has hardened by oxidation of the exothermic composition.

20. Use of the heating device of claim 2, for manufacturing a device for treating joint pain or lumbago.
